(19) United States
(12) Patent Application Publication
(10) Pub. No.: US 2022/0218915 A1
(43) Pub. Date: Jul. 14, 2022

(54) STEERABLE CATHETER OR SHEATH AND METHOD OF USE THEREOF

(71) Applicant: NORTH STAR MEDICAL INC., Montreal (CA)

(72) Inventor: Olivier BATAILLE, Lachine (CA)

(21) Appl. No.: 17/592,657

(22) Filed: Feb. 4, 2022

Related U.S. Application Data
(63) Continuation of application No. PCT/CA2020/050918, filed on Jul. 2, 2020.
(60) Provisional application No. 62/887,445, filed on Aug. 15, 2019.

(51) Int. Cl. A61M 5/36; A61M 25/01; A61M 25/00

(52) U.S. Cl. CPC A61M 5/365; A61M 25/0136; A61M 25/0147; A61M 2205/18; A61M 2205/3306; A61M 2202/0225; A61M 25/0097

(57) ABSTRACT

A steerable catheter or sheath for medical procedures; it has a shaft; one or more pull wires connected to a distal end of the shaft; and a handle connected at a distal end of the handle to a proximal end of the shaft, wherein the handle has a housing; a hub at a proximal end of the handle for connection to a valve; and a steering mechanism located closer to the proximal end of the housing than the distal end of the housing, and wherein manipulating the steering mechanism causes tension to be applied to or diminished from one or more of the one or more pull wires for steering the shaft; wherein at least a portion of the housing located between the steering mechanism and the proximal end of the housing is transparent for enabling viewing of air ingress into the steerable catheter or sheath.

The present application is a continuation application of International PCT application No. PCT/CA2020/050918 filed Jul. 2, 2020, that claims priority from U.S. provisional patent application No. 62/887,445 filed on Aug. 15, 2019, the contents of which are incorporated herein by reference.

### TECHNICAL FIELD

The present disclosure relates to steerable catheters or sheaths, and more particularly to handles for steerable catheters or sheaths for medical procedures.

### BACKGROUND

Catheters are commonly used to perform medical procedures either directly or indirectly by medical professionals. The medical professional can be located directly at the side of the patient and using a direct catheter or sheath with a handle and shaft and with a mechanical deflection mechanism in which case the deflection mechanism is directly part of the catheter handle. In the case where a medical robot is being used, the deflection mechanism may still be part of the catheter or sheath handle or the pull wire or pull wires in a case of multiple deflection or active return single deflection may be protruding out of the catheter or sheath shaft and no handle is present and the deflection wires can be attached to the robot where it can utilize its own motorized mechanism. In either case the catheter is used in various medical applications such as but not limited to interventional cardiology, electrophysiology, urology and oncology or any other minimally invasive diagnosis or therapeutic procedures. A catheter or catheter sheath used in such varying applications has a varying length shaft body having a distal end. In some cases the user or physician may be required to hold the handle and actuate the deflection mechanism with only one hand as their other hand may be performing another important part of the minimally invasive procedure. For example, in the cardiac lab, an interventional cardiologist is challenged to manage multiple devices at once such as a deflectable introducer sheath, a transseptal puncture device, a guidewire and a device delivery catheter, ablation catheter or a diagnostic visualization catheter. While some devices attempt to address this challenge with handle ergonomics, it differs from a real solution to decrease procedure time and increase positive patient outcome. Though state of the art regional and research hospitals are getting equipped with robotic solutions addressing the complexity of multiple devices handling such as the MONARCH™ or DA VINCI® robotic Platform however it is estimated that those systems, being cost prohibitive for local community hospitals, will only address 15% of the total number of minimally invasive procedures.

### SUMMARY

Current sheaths have various deflectable mechanisms with either a push button on a slider along the axis of the sheath or a rotating knob at the distal end of the handle (the distal end of the handle being the side where the handle transitions into the shaft that is inserted in the patient). In the case of the knob, the user needs to actuate or rotate the wheel typically with his or her thumb and index, with one hand. Since he or she is positioned on the right side of the patient, holding the sheath handle with the left hand, designs having the knob on the proximal side of the handle are not ergonomically correct and require the user to position their left hand over the shaft and shaft strain relief and not make use of the handle or using their right hand to hold the sheath by the handle and actuate the wheel with their right thumb and index, requiring them to insert the guidewire, delivery catheter, imaging catheter or treatment catheter, with their left hand having their arms crisscrossed.

Such deflectable devices commonly have one or multiple deflection pull wires running alongside the sheath or catheter shaft in their own lumen within the walls of the sheath to be attached at the distal end of the shaft so that when the user actuates one of the pull wires, the tension along with the lower durometer polymer used at the tip creates the deflection desired based on the level of the wire displacement in the deflection mechanism in the handle. On the proximal end of the shaft, the pull wires exit directly outside the shaft on its side prior to the shaft termination by creating a skive hole and fishing the pull wire out of its lumen. One of the major concerns is that the wires running in lumens within the main wall of the shaft create paths that need to be kept clear of air ingress, especially in cardiovascular procedures where the device may be positioned on the left side where an air embolism would be catastrophic. As such, many regulatory agencies have issued industry standards or are expecting rigorous leak testing of such devices as the standard method to perform design verification and process validation.

In the case of such devices having a handle and deflection mechanism, it is challenging to permit the design to accommodate for the user's surgical position in reference to the patient as well as taking into consideration that specific user hands are used for specific handling of devices along with making sure that there is no air ingress into the shaft's main center lumen directly accessing the cardiovascular system. In the case of a deflectable introducer sheath, the user typically is positioned on the patient's left side and holds the introducer sheath with the left hand. Such devices are typically challenging to design and manufacture in order to accommodate current surgical techniques.

Deflectable catheters and introducer sheaths devices are challenging to design and manufacture to prevent patient adverse events. The two most challenging adverse events are profuse bleeding at the introduction site or through the lumens of the devices or air ingress into the patient's cardiovascular system through the introducer site or through the lumens of the deflectable catheter or introducer sheath.

Furthermore, it is very challenging during the build of a single or multiple deflection sheaths with pull-wire(s) to make sure that at the exit holes of the pull wires the skiving does not result in an air ingress point.

A catheter or deflectable sheath is typically constructed out of polymer extrusions with varying durometers so that once constrained in a confined space only the tip with a lower durometer combination will deflect radially towards the pull wire. Deflectable catheters and introducer sheaths are manufactured via a reflow process where small polymer extrusions with each different durometers along with liners, lumen tubes, marker bands, pull wires, pull wire rings, braided wires are assembled onto a reflow mandrel and the polymers fused together securing all the other components of the shaft. In the case of deflectable devices, channels also called lumens are required to guide and allow movement for the pull wires to go from the handle deflection mechanism to the tip of the device where they are attached, when pulled the device deflects while the pull wire moves longitudinally along the pull wire lumen. In the case of catheters requiring guidewire placement, the device also has a central lumen to allow for a guidewire to slide through and being the guide to where the device needs to be as the guidewire was placed first. Having a construction with so many axial lumens and constructed with so many small polymers extrusions and components, it is challenging to keep the shaft leak proof so that bodily fluids do not escape rapidly with unintended flow paths or air ingress into the patient due to cross talk between lumens or due to voids in the reflow joints.

Therefore it would be advantageous to have a deflection mechanism combined with leak detection visualization and improved leak prevention that is better suited for the procedures with an air ingress visualization possibility so that the user may properly be positioned and have better use of his hands while being able to see a leak in progress giving him enough time to react, protecting the patient and preventing an air embolism. Such a sheath or catheter handle design with its deflection mechanism located near, e.g., the ⅔ proximal end of the handle right before the clear valve body assembly is designed to be part of the handle and its ergonomic curvature. As such, this would allow the user located on the patient's right side to hold and manipulate the sheath handle and deflection mechanism with the left hand and allow the user's right hand to be free to handle other devices such as but not limited to a guidewire, other catheters or any other devices. The hemostatic valve body can be increased in size to become part of the handle and also provide a viewing opportunity to see what can be passed through the hemostatic valve.

By providing the rotary wheel deflection mechanism near the proximal end of the sheath or catheter handle along with an external, extended and grown hemostatic valve body, the interventional cardiologist, electro-physiologist or surgeon (user) can use its left hand to hold the sheath and its right hand to feed a catheter or guidewire through the introducer sheath while on the patient's right side.

Additionally, other sections of the handle can be made clear to allow the user to view other aspects of the device that may be of interest such as observing the deflection mechanism which can allow the user to immediately know how much of an angle the deflection is by the location of the carriers. It can be further advantageous to be able to see the area around the skive holes where the pull wires exit the shaft into the handle.

Furthermore, all the components that can be made transparent, including the shaft itself so that the user has confidence in the device and does not interpret dark or opaque handles to be shields so that the user does not question the reliability of the device.

With the shaft having the ability to be transparent, the user can see the skiving holes and observe if there is air ingress or fluid egress.

To provide an additional air tight measure, a containment box with “o” rings around each end of the shaft where the skiving holes are and around the pull wires themselves can be implemented in a clear material either in a clam shell design, 3D printed or conventionally machined, optionally followed with a vapor polishing treatment to render the box transparent.

To provide better sealing transition around devices fed through the hemostatic valve, a clear donut shaped balloon filled with biocompatible fluid such as but not limited to sterile saline or sterile water for injection where the viewing window is used to detect air ingress. The balloon can be inflated or deflated with a liquid filled syringe at the proper time to better transition from a large diameter device such as a device delivery catheter to a small diameter device such as a guidewire.

As such the present disclosure relates to a sheath or catheter handle having one or more clear components to allow for error visualization, detection and counter measure. By also providing a clear containment box around the pull wire exit holes, pull wire and shaft, the device would have a significant improvement in having cross talk between lumens and further prevent air ingress or fluid egress.

During the procedure, a sheath or catheter handle can be placed in a receptacle that has an image analysis based bubble recognition system comprised, e.g., of a camera or Optical Coherence Tomography apparatus where the clear portion of the Hemostatic valve assembly is in front of such camera or OCT system and the system with either a console or small onboard computer can analyze the image generated and with a software algorithm can see a luminosity or contrast difference between fluid and air. When the system detects that there are air bubbles, the system triggers an audible, visual or sensorial alert to the user and/or can trigger the actuation of a CO2 gas delivery in the focused general area of the handle and create a CO2 blanket, chasing the air and should ingress occur, it would be CO2 ingress into the sheath or catheter and if it were to enter the patient's body, CO2 is absorbed at a much faster rate than air and therefore not likely to create an embolism (brain or lung).

To further aid the user during the actuation and manipulation of the device, a motorized deflection apparatus can be added to the device to aid in freeing the user's left hand as well where the deflection can be remotely actuated via different methods such as push button remote control, foot control, voice recognition commands having multiple functions for coarse deflection forward and backward, fine deflection forward and backward, coarse return to zero deflection. The rotating wheel of the deflection mechanism may have such a shape.

A broad aspect is a steerable catheter or sheath for medical procedures including a shaft; one or more pull wires connected to a distal end of the shaft; and a handle connected at a distal end of the handle to a proximal end of the shaft, wherein the handle comprises a housing; a hub at a proximal end of the handle for connection to a valve; and a steering mechanism located closer to the proximal end of the housing than the distal end of the housing, the steering mechanism connected to the one or more pull wires, and wherein manipulating the steering mechanism causes tension to be applied to or diminished from one or more of the one or more pull wires for steering the shaft, wherein at least a portion of the housing located between the steering mechanism and the proximal end of the housing is transparent for enabling viewing of air ingress into the steerable catheter or sheath.

In some embodiments, the catheter or sheath may include a valve connected to the hub.

In some embodiments, the shaft may be made from a transparent polymer.

In some embodiments, the steering mechanism may include a deflection wheel.

In some embodiments, the entire portion of the handle between the steering mechanism and the proximal end of the handle may be transparent.

In some embodiments, the handle may include a cavity for receiving a merging portion of the shaft at the proximal end of the shaft, and wherein one or more skiving holes may be present in the merging portion, the one or more pull wires each transitioning from the shaft to the handle by passing through one of the one or more skiving holes respectively.

In some embodiments, the handle may include a containment box for encapsulating a portion of each of the one or more pull wires when exiting the shaft and passing into the handle, for air ingress prevention and leak prevention.

In some embodiments, the containment box may have a clam-shell configuration for clamping onto the merging portion of the shaft.

In some embodiments, two halves of the clam-shell configuration of the containment box may be sealed using an adhesive, chemical fusing, ultrasonic welding or laser polymer fusion.

In some embodiments, two halves of the clam-shell configuration of the containment box may be sealed using an adhesive, and wherein the adhesive is an ultraviolet-cured adhesive.

In some embodiments, the containment box may include o-rings for sealing around each of the one or more skiving holes to prevent air entry through the one or more skiving holes.

In some embodiments, the containment box may include, for each of the one or more pull wires, a channel extension for receiving within a channel of the channel extension the each of the one or more pull wires.

In some embodiments, each channel extension may include a groove for receiving an o-ring for further sealing a pull wire located within the channel of the channel extension.

In some embodiments, the catheter or sheath may include a hemostatic valve connected to the hub; and a donut-shaped balloon positioned on a side of the hemostatic valve that faces away from the handle, and wherein the balloon is filled or fillable with a saline fluid or biocompatible fluid to prevent air ingress into the catheter.

Another broad aspect is a system for preventing air ingress when performing a medical procedure comprising the catheter or sheath as defined herein; and an air detection sub-system positionable in proximity of the transparent portion of the catheter or sheath for detecting air ingress in the catheter or sheath, wherein the detection is performed using video picture analysis or optical coherence tomography.

In some embodiments, the system may include a carbon dioxide blanketing apparatus for flooding the handle of the catheter or sheath with carbon dioxide upon the air detection sub-system detecting air ingress in the handle of the catheter or sheath.

In some embodiments, the air detection sub-system may include an alert system for alerting the user when air ingress is detected.

Another broad aspect is a system for remotely controlling the handle of the catheter or sheath as defined herein, the system including the catheter or sheath as defined herein, wherein the steering mechanism of the catheter or sheath comprises a wheel with an uneven surface pattern; a docketing handle support for receiving the handle of the catheter or sheath, the support comprising an actuating system with one or more gears that are positioned to align with the wheel of the catheter or sheath when the handle is received in the support, wherein turning of the one or more gears causes movement of the wheel of the catheter or sheath; a power source connected for providing power to the actuating system; a user input interface; and a controller that is configured to receive user input provided at the user input interface, and generates commands for controlling the actuating system based on the user input for steering the catheter or sheath by causing the wheel of the catheter or sheath to turn.

In some embodiments, the user input interface may be at least one of a microphone, a mouse of a computing device, a keyboard of a computing device and a touchscreen.

In some embodiments, the uneven surface pattern of the wheel of the catheter or sheath may be as a result of knurling.

Another broad aspect is a method of detecting air ingress during a medical procedure performed on a subject including detecting air ingress in a handle of the catheter or sheath, that is used for the medical procedure, through a transparent portion of the handle of the catheter or sheath, wherein a shaft of the catheter or sheath is inserted into the patient.

In some embodiments, the transparent portion may be located between a steering mechanism located on the handle and a proximal end of the handle where a hub connected to a valve is located.

In some embodiments, the detecting may be performed using video picture analysis or optical coherence tomography.

In some embodiments, the method may include flooding the handle of the catheter or sheath with carbon dioxide upon the detecting air ingress.

In some embodiments, the method may include alerting a user of the catheter or sheath upon the detecting of air ingress.

In some embodiments, the alerting may be performed by sounding an alarm.

In some embodiments, the alerting may be performed through a visual alert appearing on a display of a computing device, the computing device receiving a wireless signal via a short-ranged wireless translation upon the detecting of air ingress.

In some embodiments, the proximal shaft is terminated directly into a tight tolerance receptacle cavity and UV epoxy or laser welding is used to terminate the shaft directly into the valve body as one of the last assembly steps and the transparent body also allows for production in process inspection of the bonding or welding quality.

Another broad aspect is steerable catheter or sheath for medical procedures comprising a shaft; one or more pull wires connected to a distal end of the shaft; and a handle connected at a distal end of the handle to a proximal end of the shaft, wherein the handle comprises: a housing; a hub at a proximal end of the handle for connection to a valve; a steering mechanism, the steering mechanism connected to the one or more pull wires, and wherein manipulating the steering mechanism causes tension to be applied to or diminished from one or more of the one or more pull wires for steering the shaft; a cavity for receiving a merging portion of the shaft at the proximal end of the shaft, and wherein one or more skiving holes are present in the merging portion, the one or more pull wires each transitioning from the shaft to the handle by passing through one of the one or more skiving holes respectively; and a containment box for encapsulating a portion of each of the one or more pull wires when exiting the shaft and passing into the handle.

In some embodiments, the containment box may have a clam-shell configuration for clamping onto the merging portion of the shaft.

In some embodiments, two halves of the clam-shell configuration of the containment box may be sealed using an adhesive, chemical fusing, ultrasonic welding or laser polymer fusion.

In some embodiments, two halves of the clam-shell configuration of the containment box may be sealed using a chemical process or a thermal process.

In some embodiments, two halves of the clam-shell configuration of the containment box may be sealed using an adhesive, and wherein the adhesive may be an ultraviolet-cured adhesive.

In some embodiments, the containment box may include o-rings for sealing around each of the one or more skiving holes to prevent air entry through the one or more skiving holes.

In some embodiments, the containment box may include, for each of the one or more pull wires, a channel extension for receiving within a channel of the channel extension the each of the one or more pull wires.

In some embodiments, each channel extension may include a groove for receiving an o-ring for further sealing a pull wire located within the channel of the channel extension.

### BRIEF DESCRIPTION OF THE DRAWINGS

The invention will be better understood by way of the following detailed description of embodiments of the invention with reference to the appended drawings, in which:

FIG. 1 is a drawing of a cross section of an exemplary handle assembly of an exemplary deflectable sheath or catheter cross section in accordance with the present disclosure;

FIG. 2 is a drawing of an exemplary deflectable sheath or catheter in accordance with the present disclosure;

FIG. 3 is a drawing of an exemplary deflectable sheath or catheter handle cross section in accordance with the present disclosure;

FIG. 4A is a drawing of an exemplary pull wire exit hole and shaft termination containment box in accordance with the present disclosure;

FIG. 4B is a drawing of a cross section of an exemplary pull wire exit hole and shaft termination containment box in accordance with the present disclosure;

FIG. 5 is a blown-up assembly drawing of an exemplary containment box of an exemplary handle of an exemplary catheter or sheath in accordance with the present disclosure;

FIG. 6 is a drawing of an exemplary sealing balloon positioned right after the hemostatic valve in accordance with the present disclosure;

FIG. 7 is a drawing of an exemplary catheter or sheath air ingress counter measure in accordance with the present disclosure;

FIG. 8 is a drawing of an exemplary motorized system to actuate the deflection mechanism in accordance with the present disclosure; and

FIG. 9 is a block diagram of an exemplary system for controlling a handle of a catheter or sheath and for detecting the presence of air in a handle of a catheter or sheath.

### DETAILED DESCRIPTION

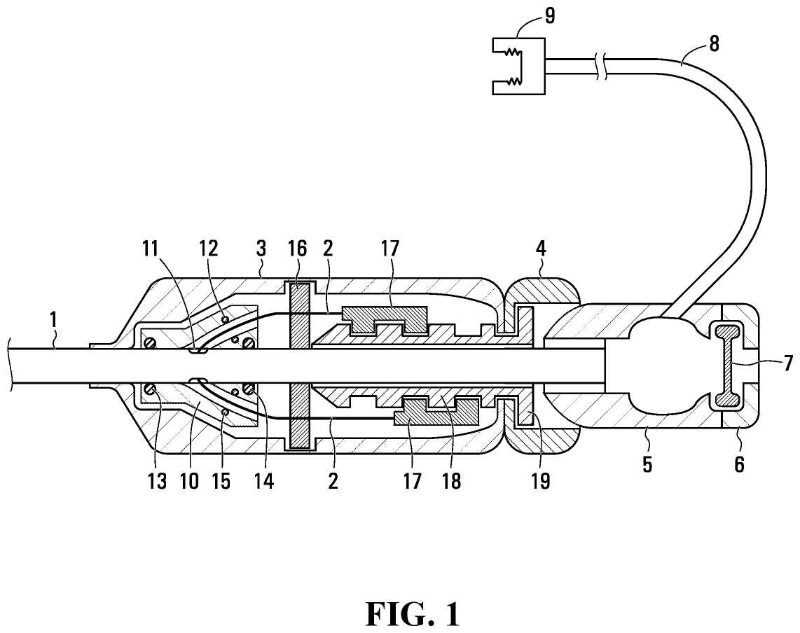

Referring now to FIG. 1, an exemplary deflectable sheath or catheter including a shaft 1 including a steering deflection mechanism with one or more pull wires 2 is shown. The pull wires 2 exit the catheter or sheath shaft 1 through skiving holes 4. The pull wires 2 are fished out of the shaft assembly by skiving with a blade or laser right above the lumen of the pull wires 2. The location of exiting pull wires 2 and skiving holes 4 is encapsulated with a leak containment box 10, sealing around the shaft with a containment box main shaft o-ring distal 13 and a containment box main shaft o-ring proximal 14 and around the pull wires 2 with one or more containment box pull wire o-rings, the number of which matching the number of pull wires. The pull wires 2 pass through the worm screw traveler stop to be then secured via welding fusing or a set screw to the worm screw mechanism carrier 17. The deflection knob 4 is attached, eliminating all degrees of freedom in relation to the worm screw 18 with press fitted pins referred to as worm screws to deflection knob attachments 19. Manually turning the deflection knob 4, the worm screw 18 is rotating in the same direction as the knob screw 4. By rotating the worm gear 18, the worm screw mechanism carriers 17 are translating either towards the proximal section of the handle 26 or the distal portion of the handle. As there is a sliding channel in the handle outer shell 3 where each of the worm screw mechanism carriers 17 are placed inside the handle, the worm screw mechanism carrier can only translate towards the proximal end 26 of the handle from the distal end of the handle 25. As each of the worm screw mechanism carriers 17 are moving in opposite directions, this system both acts as a double deflection mechanism and as an active deflection return to enable the shaft 1 to go back to perfectly straight in order to remove it from the human body cavity or vessel it may be in. The proximal end of the shaft 1 is terminated by being secured with a fastener such as an adhesive, epoxy, using ultrasonic welding or laser welding, etc., to the clear hemostatic valve body 5. The clear hemostatic valve body 5 is coupled with the clear hemostatic valve body end cap 6 to encapsulate the hemostatic valve 7 forming the proximal portion of the handle 26. The proximal portion of the handle is injection molded in a clear polymer in order for the user to be able to see that only fluid from the side port tubing 8 and side port luer hub 9 is present, using for example saline, sterile water for injection and any drugs that the user wants to inject into the patient such as heparin, commonly used in order to reduce the probability of clot formation due to foreign bodies present in the cardiovascular system. A catastrophic ingress to prevent is the ingress of air as it could cause an air embolism. As such it is important to have the proximal portion of the handle 26 adapted to allow a user to distinguish between fluid and air presence in the handle. Components of the handle proximal section can also be machined or injection-molded and, in some examples, vapor polished thereafter in order to increase visibility through the components of the handle proximal portion 26.

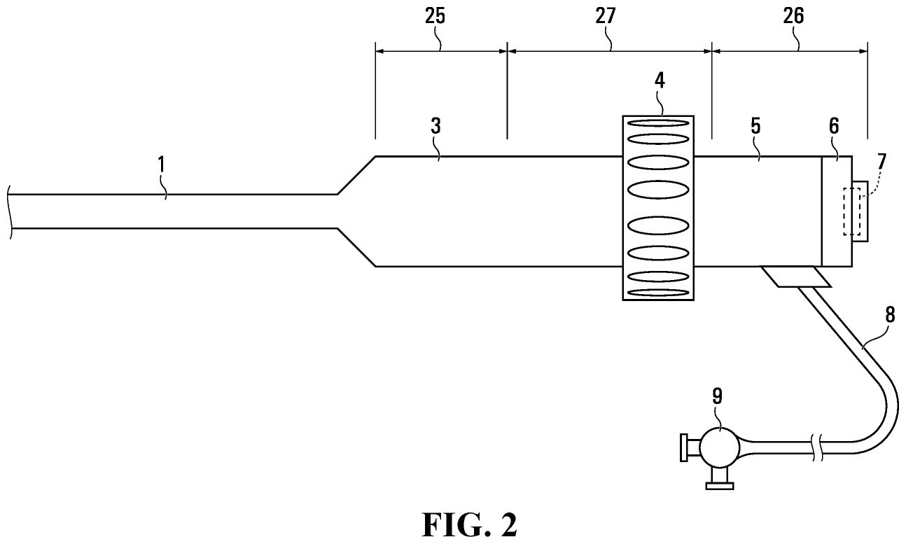

Referring now to FIG. 2, an exemplary deflectable sheath or catheter including a shaft 1 including a steering deflection mechanism is shown. The shaft passes through the handle's outer shell 3, then moving from handle distal 25 to handle proximal 26, the deflection knob 3 is located in the handle central 27 location. Immediately after the deflection knob 4 is the clear hemostatic valve body fused to the hemostatic valve end cap 6 where the hemostatic valve 7 is contained. A side port tubing 8 ending with a side port luer 9 allows the injection of fluids or drugs or flushing of the device.

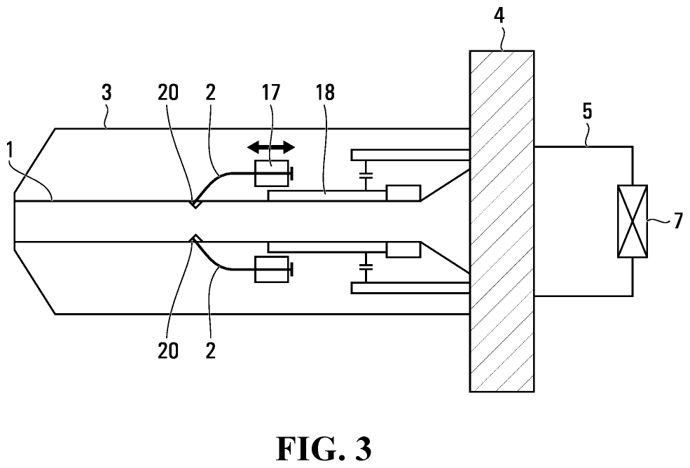

Referring now to FIG. 3, an exemplary deflectable sheath or catheter including a shaft 1 including a steering deflection mechanism with pull wires 2 is shown. The pull wires 2 exit the shaft 1 of the catheter or sheath through skiving holes 4. The pull wires 2 may be fished out of the shaft assembly by skiving with a blade or laser right above the lumen of the pull wires 2. The pull wires 2 pass through the worm screw traveler stop to be then secured via, e.g., welding fusing or a set screw to the worm screw mechanism carrier 17. The deflection knob 4 is attached, eliminating all degrees of freedom in relation to the worm screw 18 with press fitted pins referred to as worm screw to deflection knob attachments 19. Manually turning the deflection knob 4, the worm screw 18 is rotating in the same direction as the knob screw 4. By rotating the worm gear 18, the worm screw mechanism carriers 17 are translating either towards the proximal section of the handle 26 or the distal portion of the handle. As there is a sliding channel in the handle outer shell 3 where each of the worm screw mechanism carrier 17 are placed inside the handle, the worm screw mechanism carrier can only translate towards the proximal end of the handle 26 from the distal end of the handle 25. As each of the worm screw mechanism carrier 17 are moving in opposite directions, this system both acts as a double deflection mechanism and an active deflection return to enable the shaft 1 to go back to perfectly straight in order to remove it from the human body cavity or vessel as the case may be. It will be understood that other mechanisms than the one disclosed in FIG. 3 for steering the end of the distal end of the shaft by applying or removing tension from one or more of the pull wires may be used without departing from the present teachings.

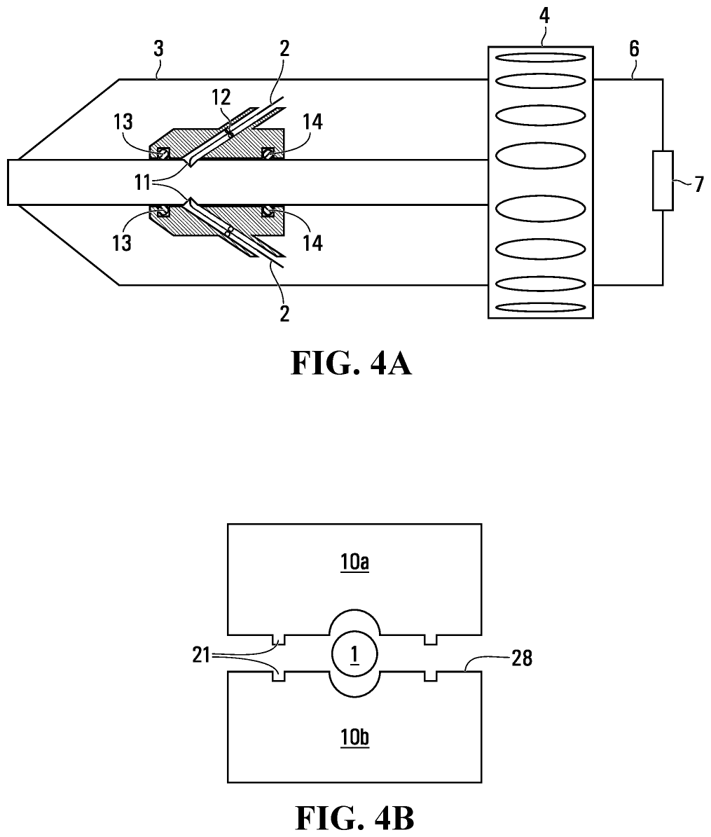

Referring now to FIG. 4A and FIG. 4B, an exemplary deflectable sheath or catheter including a shaft 1 including a steering deflection mechanism with pull wires 2 is shown. The pull wires 2 exit the catheter or sheath shaft 1 through skiving holes 4. The pull wires 2 are fished out of the shaft assembly by skiving with a blade or laser right above the lumen of the pull wires 2. The exiting pull wires 2 and skiving holes 4 are encapsulated with a leak containment box 10, sealing around the shaft with a distal containment box main shaft o-ring 13 and a proximal containment box main shaft o-ring 14 and around the pull wires 2 with one or more containment box pull wire o-rings, where the number of which may match the number of pull wires. The containment box 10 can be made in a clam shell fashion having an upper half 10a and lower half 10b that are then put together with a fastener such as an adhesive, glue, epoxy, chemical fusing, laser welding or ultrasonic welding 28, etc. The assembly of the two halves 10a and 10b of the clam shell are aided by the presence of a lip or tong and groove design that match.

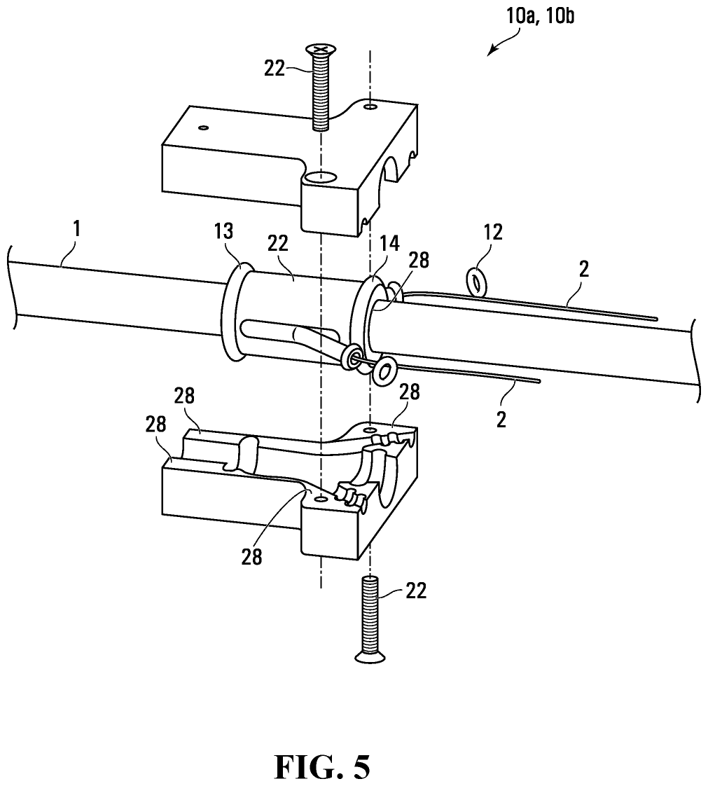

Referring now to FIG. 5, an inside view of a portion of an exemplary deflectable sheath or catheter including a shaft 1 including a steering deflection mechanism with pull wires 2 is shown. The pull wires 2 exit the catheter or sheath shaft 1 through skiving holes 4. The pull wires 2 are fished out of the shaft assembly by skiving with a blade or laser right above the lumen of the pull wires 2. The exiting pull wires 2 and skiving holes 4 are encapsulated with a leak containment box 10 (e.g. composed of the two halves 10a, 10b) sealing around the shaft with a distal containment box main shaft o-ring 13 and a proximal containment box main shaft o-ring 14 and around the pull wires 2 with one or more containment box pull wire o-rings, where the number of which may match the number of pull wires. The containment box 10 can be made in a clam shell fashion having an upper 10a and lower 10b that are then put together with a fastener such as an adhesive, glue, epoxy, chemical fusing, laser welding or ultrasonic welding 28, etc. The assembly of the two halves 10a and 10b of the clam shell may be aided by the presence of a lip or tong and groove design that can match. To further improve the seal achieved by the leak containment box 10, its two halves 10a, 10b may be joined together and in addition screwed together with two or more containment box assembly screws 22 providing a constant force and sealing pressure on the o-rings and having, e.g., the adhesive cure while the two sections are joined together by the screws 22, the two halves pressing against one another as the fastener 28 is cured or is applied.

As shown in FIG. 5, the containment box 10 may also include channel extensions 50 for receiving the pull wires. There may be a channel extension 50 that defines a tubular channel for each of the pull wires, where the pull wire pass through the channel extension 50. Within a containment box 10, there may be one or more grooves 51 for receiving o-rings for further sealing the pull wires located within the channels of the channel extensions 50 and the containment box 10.

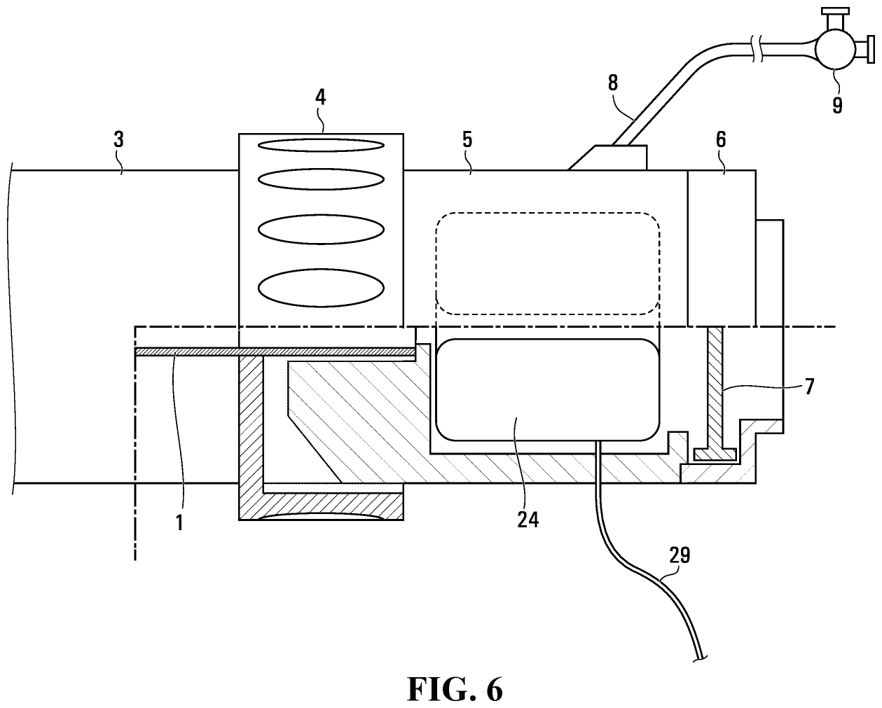

Referring now to FIG. 6, a central section and proximal section of an exemplary handle for an exemplary catheter or sheath is shown. The proximal end of the shaft 3 is terminated by being secured with a fastener, e.g. an adhesive, epoxy, using ultrasonic welding, laser welding, etc., to the clear hemostatic valve body 5. The clear hemostatic valve body 5 is coupled with the clear hemostatic valve body end cap 6 to encapsulating the hemostatic valve 7 forming the proximal portion of the handle 26. The proximal portion of the handle 26 may be injection molded in a clear polymer in order for the user to be able to see that only fluid from the side port tubing 8 and side port luer hub 9 is present, using for example saline, sterile water for injection and any drugs that the user want to directly inject into the patient such as heparin, commonly used in order to reduce the probability of clot formation due to foreign body present is the cardiovascular system. A catastrophic ingress to prevent is the ingress of air as it could cause an air embolism. As such it is important to have the proximal portion of the handle 26 be able to allow a user to distinguish between fluid and air presence in the handle. Components of the handle's proximal section 26 can also be machined or injection-molded and, in some examples, vapor polished thereafter in order to increase visibility through the components of the handle proximal portion 26. Inside the clear hemostatic valve body, a hemostatic sealing balloon is placed during the assembly and connected to the hemostatic valve injection tube. Once fully assembled, the user can easily inflate or deflate the balloon with fluid to seal around a catheter, guide wire or delivery shaft in order to further prevent air ingress through the hemostatic valve 7.

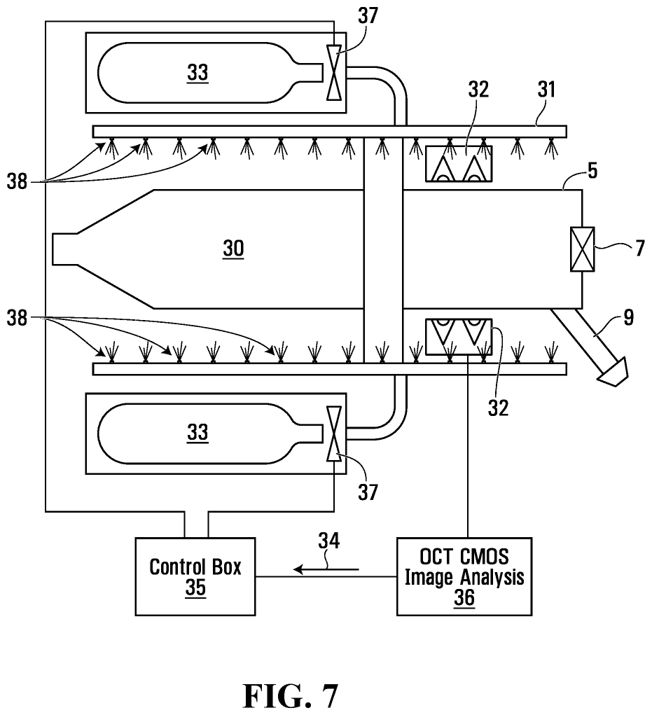

Referring now to FIG. 7, a catheter or deflectable sheath 30 inserted in a receptacle 31 where an air detection device such as a CMOS camera or an optical coherence tomography apparatus 32 is placed right around the clear valve body 5 and the camera or optical coherence tomography apparatus 32 can detect changes in contrast between liquid and air in the case of the CMOS camera and can have a different OCT light source reflection between air and fluid and can therefore be programmed to produce an alarm (e.g. a sound, a light, a vibration, etc.) for the user when a difference is recognized by the system with its OCT or CMOS image analyzing computer 36 which can then trigger and send a signal 34 to the control box where normally closed valves 37 can be signaled to open and release CO2 gas from the CO2 cartridges contained within the receptacle 31. The gas can be grossly released within the vicinity of the handle so that a CO2 blanket is created or the CO2 gas can be released through multiple nozzles 38 that are part of the receptacle 31.

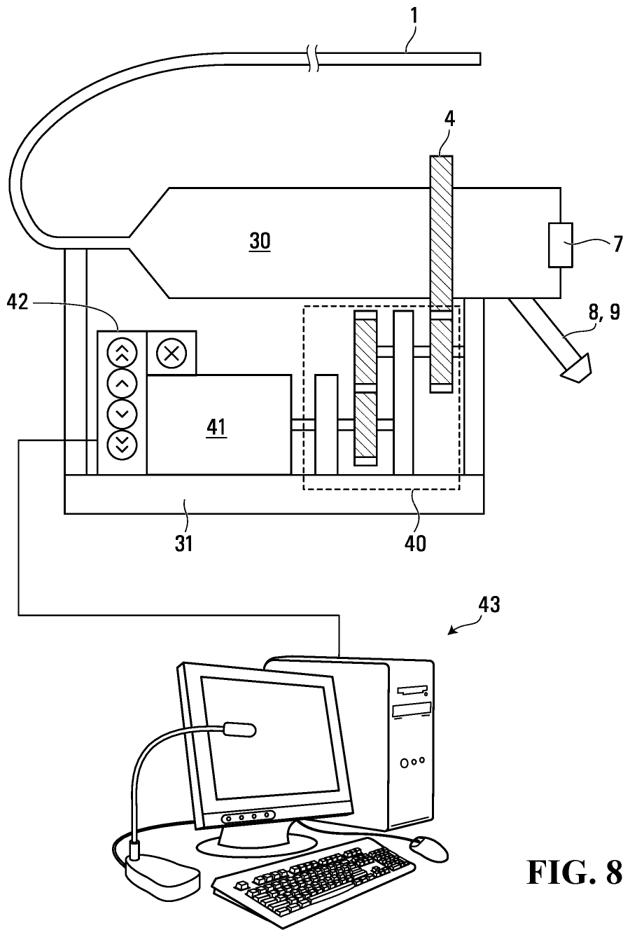

Referring now to FIG. 8, an exemplary catheter or deflectable sheath 30 inserted in an exemplary receptacle 31 where the shape of the handle outer shell 3 may be used to position the handle in the receptacle 31. Once the deflectable catheter or sheath is place in the receptacle, the deflection knob or wheel pairs with coupling gear 39, the gear 39 (e.g. with a knurled surface) being part of a gear box/mechanism 40. The gear box mechanism 40 can be engaged, turning with its connection to a motor 41. The motor may be controlled by a computing device 42 (e.g. control box) that includes multiple buttons to control the motor to turn slowly or rapidly in either direction. The motor 41 can also be controlled to turn slow or fast in either direction by a computing device 42 that is a computer with, e.g., voice recognition 43 where specific voice commands can be interpreted as instructing the motor to go clockwise slow, clockwise fast, counter-clockwise slow, counter-clockwise fast, stop, to go back to neutral position, etc. In some examples, the computer may be a remote computer.

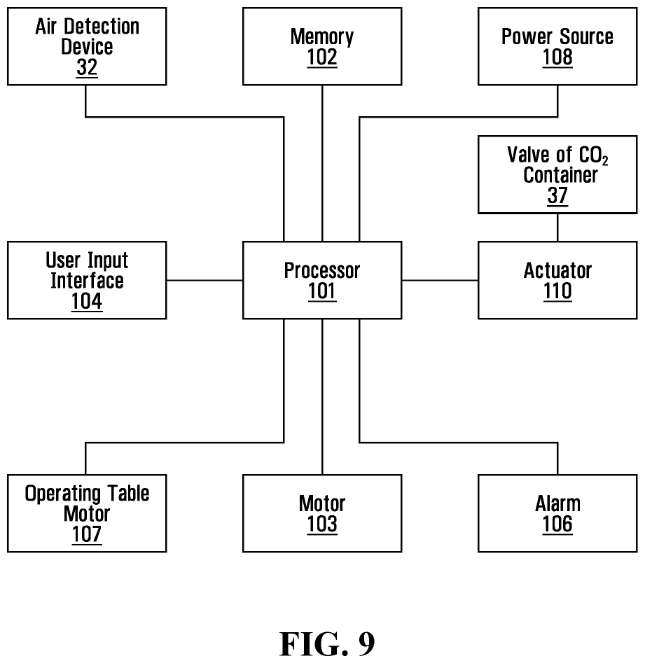

As shown in FIG. 9, the computing device 42 may therefore have a user input interface 104 (e.g. keyboard, mouse, touchpad, microphone, etc.) for receiving input from the user of the catheter or sheath system for controlling same. The computing device 42 also has a controller. The controller includes a processor 101 (e.g. single or multiprocessor) and memory 102, where the processor 101 and memory 102 are connected via a BUS. The memory 102 stores program code that, when executed by the processor 101, causes the processor 101 to carry out certain commands. For instance, the program code may be such as to cause the processor 101 to issue certain commands to the motor 103 to cause the motor 103 to move in a certain manner as a function of the user input received at the user input interface 104 (once the processor analyzes the received input), for controlling the handle of the catheter or sheath positioned in the receptable 31. The connection between the computing device 42 and the motor or the receptable 31 may be wired or wireless. The computing device 42 may also have a power source 108 (e.g. a battery, power outlet, etc.)

In some examples, the computing device 42 may also have the functionality of computer 36 to detect the presence of air as a function of data received from the air detection device 32.

In some examples, computer device 42 may also be connected to actuator 110 for opening or closing the valve(s) of the CO2 containers as a function of the detection of air in the catheter or sheath.

In some examples, the computing device 42 may also be connected to an alarm 106 for alerting the user of the catheter or sheath as to the presence of air therein.

In some embodiments, the computing device 42 may also be connected to a motor of an operating table 107 on which the patient is laid. Upon the detection of air, the computing device 42 may issue one or more commands to the operating table motor 107 to cause the operating table to tilt such that the toes of the patient are located above the head of the patient, such that the air is encouraged to travel up towards the toes of the patient.

Exemplary Method of Detecting Air Ingress in a Handle of A Catheter or Sheath:

The present disclosure also relates to an exemplary method of detecting air ingress in a catheter or sheath, namely through a transparent portion located in the handle of the catheter or sheath.

The handle may have a transparent portion located near a proximal end of the handle, between the steering mechanism (e.g. wheel mechanism) of the handle and the proximal end of the handle which may have a hub for connecting to a valve (e.g. hemostatic valve), as explained herein.

During the course of a medical procedure, air may be detected in the transparent portion of the handle. Either the user, or an air detection device as explained herein, may detect the presence of air in the handle. The detection of air indicates that the patient is in danger, as the air may cause an air embolism in the patient.

As such, once air is detected, a command may be sent by the air detection device (or a computing device connected to the air detection device) to an actuator of a valve that opens or closes a CO2 container. The command may cause the actuator to open the valve, resulting in the release of CO2 from its container, where the CO2 may flood the handle of the catheter or sheath. The CO2 would then enter the handle instead of air, the CO2 more readily absorbed by the bloodstream of the patient than the air.

Once air is detected, a command may be sent by the air detection device (or a computing device connected to the air detection device) to an alarm to alert the user (e.g. medical practitioner) of the presence of air. The alarm may be a sound, a light, a tactile sensation (e.g. vibration), etc.

In some embodiments, once air is detected, a command may be sent by the air detection device (or a computing device connected to the air detection device) to a controller of an operating table, causing the operating table to tilt such that the patient is at an angle where the toes of the patient are elevated above the patient's head, encouraging the air to flow towards the toes of the patient.

Although the invention has been described with reference to preferred embodiments, it is to be understood that modifications may be resorted to as will be apparent to those skilled in the art. Such modifications and variations are to be considered within the purview and scope of the present invention.

Representative, non-limiting examples of the present invention were described above in detail with reference to the attached drawing. This detailed description is merely intended to teach a person of skill in the art further details for practicing preferred aspects of the present teachings and is not intended to limit the scope of the invention. Furthermore, each of the additional features and teachings disclosed above and below may be utilized separately or in conjunction with other features and teachings.

Moreover, combinations of features and steps disclosed in the above detailed description, as well as in the experimental examples, may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

### REFERRING TO THE EXEMPLARY FIGURES

1. Shaft, from sheath or catheter
2. Pull-wires
3. Handle outer shell covering the deflection mechanism
4. Deflection knob or thumb wheel
5. Clear hemostatic valve body
6. Clear hemostatic valve body end-cap
7. Hemostatic valve
8. Side port tubing
9. Side port luer hub
10. Leak containment box around the pull wire exit holes (can be in clam shell 10a and 10b)
11. Skive holes
12. Containment box pull wire “O” ring
13. Containment box main shaft “O” ring distal
14. Containment box main shaft “O” ring proximal
15. Containment box pull wire “O” ring
16. Worm screw traveler stop
17. Worm screw mechanism carrier
18. Worm screw
19. Worm screw to deflection know attachment
20. Skive holes exit
21. Tongue and groove feature
22. Containment box assembly screw
23. Containment box inner body
24. Hemostatic sealing balloon
25. Handle distal
26. Handle proximal
27. Handle central
28. Glue, Epoxy, Chemical fusing, laser welding or ultrasonic welding
29. Hemostatic balloon injection tube
30. Catheter or deflectable sheath
31. Receptacle
32. CMOS camera or an optical coherence tomography apparatus
33. CO2 cartridge
34. Signal
35. Control Box
36. OCT or CMOS image analyzing computer
37. normally closed valves
38. multiple nozzles
39. Coupling gear
40. Gear box/mechanism
41. Motor
42. Control box
43. Computer with voice recognition
50. Channel extensions
51. Grooves

What is claimed is:

1. A steerable catheter or sheath for medical procedures comprising:
a shaft;
one or more pull wires connected to a distal end of said shaft; and
a handle connected at a distal end of said handle to a proximal end of said shaft, wherein said handle comprises:
a housing;
a hub at a proximal end of said handle for connection to a valve; and
a steering mechanism located closer to said proximal end of said housing than said distal end of said housing, said steering mechanism connected to said one or more pull wires, and wherein manipulating said steering mechanism causes tension to be applied to or diminished from one or more of said one or more pull wires for steering said shaft;
wherein at least a portion of said housing located between said steering mechanism and said proximal end of said housing is transparent for enabling viewing of air ingress into said steerable catheter or sheath.

2. The catheter or sheath as defined in claim 1, wherein said shaft is made from a transparent polymer.

3. The catheter or sheath as defined in claim 1, wherein said handle comprises a cavity for receiving a merging portion of said shaft at said proximal end of said shaft, and wherein one or more skiving holes are present in said merging portion, said one or more pull wires each transitioning from said shaft to said handle by passing through one of said one or more skiving holes respectively, wherein said handle further comprises a containment box for encapsulating a portion of each of said one or more pull wires when exiting said shaft and passing into said handle.

4. The catheter or sheath as defined in claim 3, wherein said containment box has a clam-shell configuration for clamping onto said merging portion of said shaft, wherein two halves of said clam-shell configuration of the containment box are sealed using one of a thermal process and a chemical process.

5. The catheter or sheath as defined in claim 3, wherein said containment box further comprises o-rings for sealing around each of said one or more skiving holes to prevent air entry through said one or more skiving holes.

6. The catheter or sheath as defined in claim 3, wherein said containment box comprises, for each of said one or more pull wires, a channel extension for receiving within a channel of said channel extension said each of said one or more pull wires.

7. The catheter or sheath as defined in claim 6, wherein each channel extension comprises a groove for receiving an o-ring for further sealing a pull wire located within said channel of said channel extension.

8. A system for preventing air ingress when performing a medical procedure comprising:
the catheter or sheath as defined in claim 1;
an air detection sub-system positionable in proximity of said transparent portion of said catheter or sheath for detecting air ingress in said catheter or sheath, wherein said detection is performed using video picture analysis or optical coherence tomography.

9. The system as defined in claim 8, further comprising:
a carbon dioxide blanketing apparatus for flooding said handle of said catheter or sheath with carbon dioxide upon said air detection sub-system detecting air ingress in said handle of said catheter or sheath.

10. The system as defined in claim 8, wherein said air detection sub-system further comprises an alert system for alerting the user when air ingress is detected.

11. A system for remotely controlling said handle of said catheter or sheath as defined in claim 1, comprising:
said catheter or sheath, wherein said steering mechanism of said catheter or sheath comprises a wheel with an uneven surface pattern;
a docketing handle support for receiving said handle of said catheter or sheath, said support comprising an actuating system with one or more gears that are positioned to align with said wheel of said catheter or sheath when said handle is received in said support, wherein turning of said one or more gears causes movement of said wheel of said catheter or sheath;
a power source connected for providing power to said actuating system;
a user input interface; and
a controller that is configured to receive user input provided at said user input interface, and generates commands for controlling said actuating system based on said user input for steering said catheter or sheath by causing said wheel of said catheter or sheath to turn.

12. The system as defined in claim 11, wherein the user input interface is at least one of a microphone, a mouse of a computing device, a keyboard of a computing device and a touchscreen.

13. A method of detecting air ingress during a medical procedure performed on a subject comprising:
detecting air ingress in a handle of said catheter or sheath, that is used for said medical procedure, through a transparent portion of said handle of said catheter or sheath, wherein a shaft of said catheter or sheath is inserted into said patient.

14. The method as defined in claim 13, wherein said transparent portion is located between a steering mechanism located on said handle and a proximal end of said handle where a hub connected to a valve is located.

15. The method as defined in claim 13, wherein said detecting is performed using video picture analysis or optical coherence tomography.

16. The method as defined in claim 13, further comprising flooding said handle of said catheter or sheath with carbon dioxide upon said detecting air ingress.
